(19) United States
(12) Patent Application Publication
(10) Pub. No.: US 2024/0407888 A1
(43) Pub. Date: Dec. 12, 2024

(54) ORAL APPLIANCE FOR PROMOTION OF NASAL BREATHING DURING SLEEP

(71) Applicant: William Laurence Oliver, Grapevine, TX (US)

(72) Inventor: William Laurence Oliver, Grapevine, TX (US)

(21) Appl. No.: 18/437,853

(22) Filed: Feb. 9, 2024

Related U.S. Application Data
(60) Provisional application No. 63/507,144, filed on Jun. 9, 2023.

(51) Int. Cl. A61C 5/00; A61C 7/08; A61C 9/00

(52) U.S. Cl. CPC A61C 5/007; A61C 7/08; A61C 9/0006; A61C 9/0053

(57) ABSTRACT

A custom-fitted oral appliance designed without vents to restrict oral breathing is disclosed. When wearing the oral appliance, a wearer (or patient) is encouraged by the appliance to breathe through his/her nose when asleep. The custom-fitted oral appliance is uniquely designed to restrict oral breathing (of the wearer) when asleep, for promoting an optimal breathing routine whenever asleep, which is nasal breathing. The appliance is designed to place a wearer's lower jaw in a relaxed position, keeping it there when asleep.

### CROSS-REFERENCE TO RELATED APPLICATION

This application claims the benefit of priority of U.S. provisional application No. 63/507,144, filed 9 Jun. 2023, the contents of which are herein incorporated by reference.

### BACKGROUND OF THE INVENTION

The present subject matter for an oral appliance is more particularly directed to an oral appliance that promotes nasal breathing and suppresses oral breathing during sleep.

As we all know, humans can breathe both through the mouth and through the nose. Because large volumes of air can be rapidly exchanged, mouth breathing is necessary during periods of high oxygen demand, such as during exercise or exertion. Sleep is a period when the body enters a restful state and has much lower oxygen demand. During sleep, experts believe that people should ideally breathe through their nose. Breathing, however, usually follows the path of least resistance, which is through an open mouth, rather than the nose.

There is a large body of scientific research that shows that nasal breathing is the optimal breathing method during sleep. Unlike the mouth, the human nose is highly efficient in heat or cold dissipation, air filtration and moisturization. During sleep, everyone cycles about every 90 minutes between Rapid Eye Movement (REM) sleep and Non-Rapid Eye Movement (NREM) sleep. During REM sleep, people are dreaming, and their brain wave activity is like that of a person who is awake. Except for the eye muscles, the voluntary muscle function during this period is temporarily disabled and the entire body experiences a loss of muscle tone. This temporary disablement is postulated by sleep researchers to be a protective mechanism to prevent the dreamer from acting out their dreams and potentially injuring themselves. It is during this time that the jaw muscles relax allowing the mouth to open and the tongue to fall back into the throat to potentially block the oral airway.

When a person is sleeping and enters REM sleep, the jaw muscles relax and lose their tone. This allows the mandible to fall into a relaxed or “retruded” (posterior displacement) position, the teeth then separate, and the mouth opens. This space between the teeth during REM sleep differs naturally from person to person and is usually between 5 millimeters (mm) to 15 mm.

Snoring, a common problem among people of all ages, is caused by the vibration of the soft tissues of the uvula, soft palate, and adjacent structures and may result from the restriction of airflow through the pharyngeal passageway. For the most part, snoring is harmless and presents no real physical threat to the snorer. Yet, this affliction can result in no end of aggravation and annoyance directed at the snorer from all those that sleep within earshot of the snorer.

Moreover, certain people suffer a more advanced “condition” commonly known as sleep apnea, which may cause a sleeper to stop breathing during sleep, resulting in a life-threatening situation. Both afflictions can be reduced—or even prevented—by decreasing the restriction of the pharyngeal passageway which might occur during sleep.

The tongue, lips, and other components of the mouth of a person exert significant influences on breathing and ancillary functions of the body. Efficiency of breathing and air flow within the mouth and other breathing passages maximizes physiological functions, such as athletic activity and nighttime breathing. Studies suggest that unimpeded air flow during sleep is a characteristic of those who don't snore when asleep. Tissues of structure associated with the mouth and breathing passages can deform over time due to age or due to various congenital or induced conditions of infirmity. These tissues may become displaced in response to certain conditions noted and may even interfere with breathing.

Prophylactic devices for reversing adverse effects of displaced tissue have been proposed in the prior art. In this regard, the benefit of supporting the tongue within the mouth has long been recognized as leading to improvement of various debilitating conditions. Devices worn in the mouth to affect position of the tongue have been proposed as leading to elimination of snoring, improvement in breathing, and various other benefits.

U.S. Pat. No. 6,055,986 to Meade discloses an apparatus for intra-oral use. The apparatus includes a framework having an upper curved platform and a lower curved platform. Each platform is defined by two walls extending from and separated by a floor. A material bonded to the upper and lower curved platform is adapted to be molded in a shape suitable not only for snugly receiving the maxillary teeth of a user but also for loosely receiving the mandibular teeth of the user when the mouth is closed in a normal closing arch, and a cavity—between the upper and lower platforms—is adapted to receive the tongue of the user. The apparatus may also include an adjustment mechanism to adjust the positioning of the upper platform with respect to the lower platform.

U.S. Pat. No. 6,129,084 to Bergersen discloses an oral appliance for repositioning the mandible anterior to the maxillary teeth, to open the oral and pharyngeal passageways of the user, to prevent snoring and sleep apnea. The appliance includes two U-shaped plates joined to form a hinge. An upper plate has a labial-buccal wall but no lingual wall, to allow anterior positioning of the tongue. A lower plate has both a labial-buccal wall and a lingual wall. The walls, pliable to reduce pressure on teeth, vary in height and thickness.

U.S. Pat. No. 6,295,988 to Sue discloses an oral device to hold the tongue pressed against the roof of a mouth, to cause suction when closing lips and encouraging breathing through a nose. The device includes a U-shaped channel for engaging the upper teeth of a user. The channel includes a vertical wall. A platform projects horizontally and inwardly from the channel. The platform, located at bottom surfaces of the upper teeth, supports the tongue. The platform has concave indentations conforming to the individual teeth of a user. The platform has a flat bottom surface, at least one drainage opening to enable saliva to pass to the bottom of the mouth, and another opening to the rear of the device.

U.S. Pat. No. 7,004,172 to Zacco discloses a mouthpiece body made of thermoplastic material, shaped complementary to the dental arch of a person, to reduce snoring. The body includes a posterior end having two spaced-apart members positioned toward the back of the dental arch of a person when properly worn, and an anterior end having an airway opening through the body. When worn properly, the anterior end supports the lips of a person in a spaced-apart arrangement, so that air flows through the airway opening.

U.S. Pat. No. 7,810,502 to Nguyen et al. discloses a “so-called” anti-snoring and obstructive-sleep-apnea device for preventing the lower jaw of a person from receding and preventing the tongue of a person from blocking the air passageway, for eliminating the person snoring. The “so-called” anti-snoring and obstructive sleep apnea device includes a first support removably retained by the upper teeth of a user and includes a second support supported by the lower teeth of a user. The device further includes an assembly retained by the first support for positioning the second support and preventing the lower jaw from receding and the tongue from blocking the air passageway of a user and to allow opening the mouth and moving the lower jaw of a user forwardly and laterally.

U.S. Pat. No. 9,545,331 to Ingemarsson-Matzen discloses an adjustable mandibular advancement device having a combination of resilient hinging, dentition engagement, and adjustability features, designed to prevent or reduce Snoring and/or Obstructive Sleep Apnea Syndrome. Adjustment is achieved by two concepts involving relative enlargement or diminution of certain members of the device, that are associated with incremental and successive advancement mechanisms of the device, either in combination or separately.

U.S. Pat. No. 10,898,369 to Farrell discloses a set of oral appliances comprising a first and at least a second oral appliance. Each (of the first and at least second oral appliances) includes a generally U-shaped appliance body including an inner wall, an outer wall, and a web interconnecting the inner wall and the outer wall. The web has a front section and two arms with trailing ends. The web includes at least one breathing hole to define a total cross-sectional area for breathing and the total cross-sectional area for breathing of the first oral appliance is larger than the total cross-sectional area for breathing of the second oral appliance. A patient wears the first oral appliance, in the first treatment stage, while sleeping for a first period. The patient wears the second oral appliance, in a second treatment stage, while sleeping for a second period.

U.S. Pat. No. 11,123,219 to Tamez et al. discloses a mouth appliance to promote nasal breathing and reduce or eliminate snoring and to help improve breathing during exercise. The mouth appliance includes upper and lower pieces, hinged at the rear, and configured to fit the mouth and teeth of a wearer. The upper and lower mouth pieces are joined to create a seal and promote nasal breathing. The frame of the appliance may be formed from a hard or semi-hard material and may be formed to the mouth of a wearer to ensure a comfortable and effective fit. The upper and lower pieces include a latching mechanism to allow for a mouth to be opened, closed, or semi-opened during wear and use of the appliance, and to allow the wearer to talk and/or drink without removing the appliance. The mouthpiece can be worn at any time, for protecting the teeth of the wearer.

There is a large body of scientific research indicating nasal breathing is an optimal way to breathe when asleep. Unlike the mouth, a human nose is highly efficient in heat or cold dissipation, air filtration, and moisturization. Mouth breathing when asleep is associated with apnea (breathing stoppage), xerostomia (dry mouth), and bad breath (halitosis). Mouth breathers are also more likely to experience tooth decay, gum diseases, digestive issues, chronic fatigue, morning headaches, and sore throats.

Yet snoring and sleep apnea are afflictions that continue to torment the general population. As can be seen, there is a need for an oral appliance that promotes nasal breathing and suppresses oral breathing during sleep in a novel and non-obvious manner relative to the assortment of devices found in the prior art.

### SUMMARY OF THE INVENTION

The present subject matter is directed to a custom-fitted oral appliance designed without vents to restrict oral breathing. When wearing the appliance, a user (or patient) is encouraged to breathe through his/her nose when asleep. The custom-fitted appliance of the present subject matter is uniquely designed to restrict oral breathing when asleep, to promote an optimal breathing routine (i.e., nasal breathing) when asleep. The appliance is designed to place the lower jaw in a relaxed position and keep it there when sleeping.

The oral appliance that will govern the breathing dynamic during sleep by preventing oral breathing. The present invention is not a Mandibular Advancement Device (MAD). MADs move the mandible (lower jaw) into an unnatural protrusive position and can cause Temporomandibular Joint (TMJ) pain and tooth movement.

MADs also promote mouth breathing.

The oral appliance embodied in the present invention is designed to attach to the teeth of the maxilla (upper jaw) and the teeth of the mandible. The appliance will hold the mandible in the optimal retruded/relaxed position for that individual and does not have vents for mouth breathing. Holding the mandible in the optimal relaxed position promotes comfortable sleep with nasal breathing and minimal TMJ stress. Therefore, the oral appliance will promote nasal breathing and suppress mouth breathing. The oral appliance comes in the form of a one-piece (unitary construction) flexible device that attaches over both the upper and lower teeth. It will prompt the user to breathe through their nose and suppress mouth breathing, since mouth breathing during sleep is associated with apnea (stoppage of breathing), xerostomia (dry mouth) and bad breath (halitosis).

The appliance is contraindicated in people with Central Sleep Apnea (CSA) and Nasal Airway Obstruction (NAO). There are several treatments for NAO, many of which can be performed by ENT specialists.

In one aspect of the present invention, oral appliance for promoting nasal breathing and for suppressing oral breathing of a wearer, the oral appliance provides the following: an upper flange defining a plurality of upper teeth impressions arranged along a lower surface portion of the upper flange, wherein the plurality of upper teeth impressions is dimensioned and shaped to snugly receive the upper teeth of the wearer; a lower flange unitary with the upper flange, wherein the lower flange defines a plurality of lower teeth impressions arranged between the flange opposite end portions along an upper surface portion of the lower flange, wherein the plurality of lower teeth impressions are dimensioned and shaped to snugly receive the lower teeth of the wearer; and a spacer unitary with the upper flange and the lower flange, wherein the spacer is dimensioned and configured for enabling an upper lip and a lower lip of the wearer to contact, thereby promoting nasal breathing and for suppressing oral breathing of the person.

The spacer is tailored, dimensioned, and sized so as not to prevent the patient from closing their lips together, while their mandible is in the retruded position, thereby minimizing secretion of saliva (drooling) and to prevent drying of the mucosal tissues (inner lining of the lips). In other words, the spacer provides the correct amount of separation of the teeth, while allowing for full lip closure; though it is to be understood that with or without the spacer, the wearer should be able to close their lips without restriction from the oral appliance. Furthermore, an upper buccal vestibule barrier extending posteriorly from the upper flange; and a lower buccal vestibule barrier extending inferiorly from the lower flange, wherein each buccal vestibule barrier is configured to occupy a buccal vestibule adjacent thereto so as to prevent passage of the airflow through the respective buccal vestibule.

In another aspect of the present invention, method of making a unitary oral appliance to promote nasal breathing and suppress oral breathing of a wearer, the method including: using a bite registration material configured for determining a separation value between a lower jaw and an upper jaw of the wearer, wherein the separation value is determined when the lower jaw and the upper jaw are in a relaxed position relative to each other; using an impression material configured or an oral scanning device configured to capture a size, a shape, and a position of each tooth in the lower and upper jaws of the wearer or using to each other; placing a model of the teeth of the wearer on a dental articulator adapted and configured to account for the separation value; and fabricating the unitary oral appliance having extensions into the buccal vestibules of the wearer to restrict oral air exchange when breathing orally, wherein the model is a virtual model, and wherein the fabricating step occurs in a dental lab.

In yet another aspect of the present invention, an oral appliance for promoting nasal breathing and for suppressing oral breathing of a wearer, the oral appliance provides the following: a unitary construction that occupies the oral cavity to prevent passage of an airflow from an external environment to the pharyngeal passageway of the wearer by way of the mouth of the wearer when the mandible is in a retruded position; a plurality of impressions within the oral applicance for receiving upper and lower teeth of the wearer; and upper and lower barriers that occupy the superior and inferior buccal vestibules, respectively, of the wearer; and further providing anteriorly protruding upper and lower spacers for abutting an inner surface of the upper and lower lips of the wearer so that a distal edge of said lips sealably contact when the mandible is in the retruded position.

These and other features, aspects and advantages of the present invention will become better understood with reference to the following drawings, description and claims.

### BRIEF DESCRIPTION OF THE DRAWINGS

FIG. 1 depicts a front perspective view of an embodiment of an oral appliance of the present subject matter, on an enlarged scale relative to a size when used by a person.

FIG. 2 is a flow chart for manufacturing an exemplary embodiment of the present invention.

FIG. 3 is a rear perspective view of an exemplary embodiment of the present invention, such that components shown on the left in FIG. 1 are on the right in FIG. 3.

FIG. 4 is a sectional view of an exemplary embodiment of the present invention, shown in use condition.

FIG. 5 is a side perspective view of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense but is made merely for the purpose of illustrating the general principles of the invention, since the scope of the invention is best defined by the appended claims.

Broadly, an embodiment of the present invention provides a custom-fitted oral appliance designed without vents to restrict oral breathing is disclosed. When wearing the oral appliance, a wearer (or patient) is encouraged by the appliance to breathe through his/her nose when asleep. The custom-fitted oral appliance is uniquely designed to restrict oral breathing (of the wearer) when asleep, and thereby promoting an optimal breathing routine whenever asleep, which is nasal breathing. The appliance is designed to place a wearer's lower jaw in a relaxed or retruded position, keeping it there when asleep.

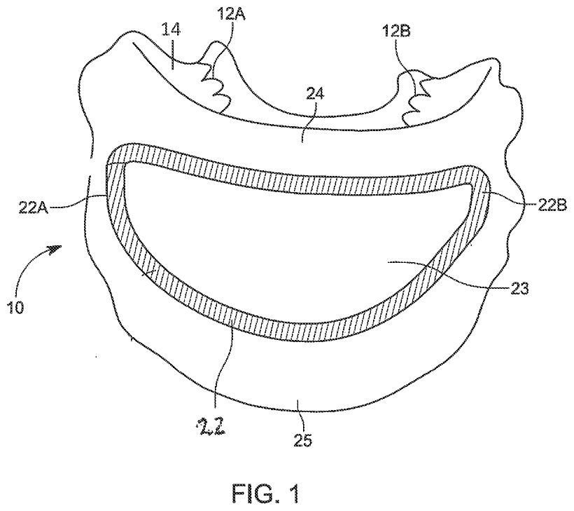

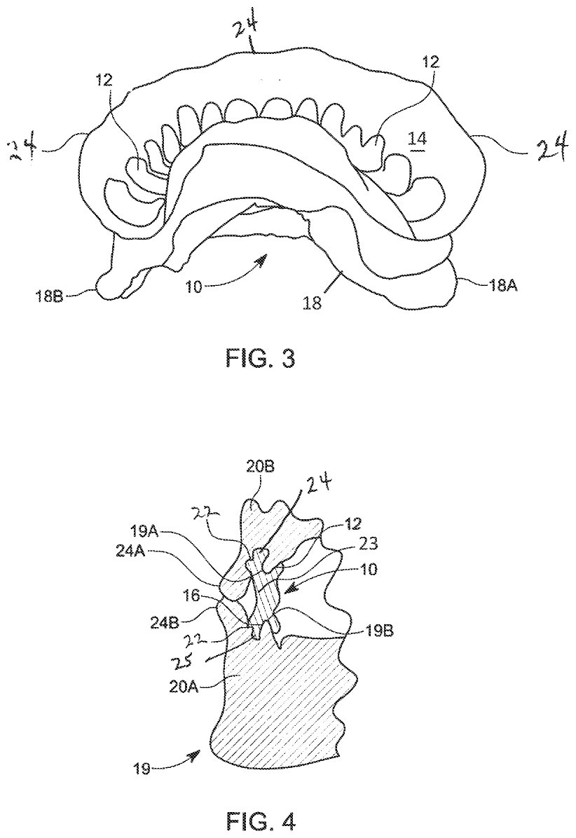

Referring initially to FIGS. 1, 3, 4, and 5, an embodiment of an oral appliance 10 of the present invention—designed to be used for promoting nasal breathing and suppressing oral breathing of a person 19—is described as follows.

The oral appliance 10 includes an upper flange 14 and a lower flange 18 spaced from the upper flange 14. The upper flange 14 includes spaced-apart opposite end portions 14A and 14B. The upper flange 14 was fabricated to define the upper teeth impressions 12 of a person 19 having upper teeth 19A. The upper teeth impressions 12are arranged between the upper flange end portions 14A and 14B along a lower surface portion of the upper flange 14. The upper teeth impressions 12 are dimensioned and configured to snugly receive-preferably customized to mesh with and frictionally engage external surface portions of—the upper teeth 19A of the wearer 19 of the oral appliance 10.

The lower flange 18, unitary with upper flange 14, includes spaced-apart opposite end portions 18A and 18B. The lower flange 18 defines the lower teeth impressions 16 of lower teeth 19B. The lower teeth impressions 16, arranged between the end portions 18A and 18B along an upper surface portion of the lower flange 18, are dimensioned and configured to snugly receive—and preferably could be customized to mesh with and frictionally engage external surface portions of—the lower teeth 19B of the wearer 19.

The relative locations and orientations of the upper and lower teeth impressions 12 and 16 are identified through utilization of an impression material, through a scanning process, or other methodology that ascertains the locations and orientations of the upper and lower teeth 19A and 1B of the wearer 19 with their lower jaw in the retruded position.

The oral appliance 10 further includes a spacer 22 unitary with the upper flange 14 and the lower flange 18. The spacer 22 of the oral appliance 10 is custom fabricated to be dimensioned and configured for enabling the upper lip 24A and the lower lip 24B of the wearer 19 to contact, whenever the oral appliance 10 is worn by the wearer 19. This contact between the upper and lower lips 24A and 24B is sufficient for them to form a seal and/or barrier to moisture entering or exiting the mouth of the wearer 19, which in turn furthers the present invention's purpose of promoting nasal breathing and suppressing oral breathing of the person 19. In conjunction with the upper flange 14 and lower flange 18, the spacers 22 prevent seepage of saliva out of the mouth during use, e.g., while sleeping. The spacer 22 may be a small semi-circular ridge which is on the labial (buccal) surface of the oral appliance 10. The spacer 22 is purposed to help prevent saliva seepage from the mouth during sleep. The spacer ridge is only about 1-2 mm in semi-circular radius. It should not interfere with the wearer's ability to have passive contact of their lips when sleeping; rather, the ridge of the spacer 22 abut or props against an inner surface of the labium/lips within the buccal cavity, thereby urging the distal edge of the upper and lower lips 19A and 19B to contact when the jaw is in the retruded position during sleep.

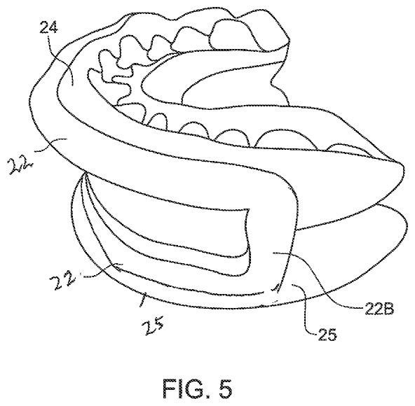

The spacer 22 ridge may define a closed, uninterrupted loop by way of side spacer ridges 22A and 22B that join the upper and lower portions of the ridge 22, as illustrated in FIG. 5.

The oral appliance 10 includes a closed wall area 23 extending between a maxilla extension/barrier 24 and a mandibular buccal extension/barrier 25. The closed wall area 23 is recessed relative to the maxilla and mandibular buccal extensions 24 and 25as well as the spacer portions 22, 22A, 22B. The closed wall area 23 is a continuous, uninterrupted surface configured to prevent any venting or exhausting of air through the oral appliance 10.

“User comfort” when using embodiments of the oral appliance of the present invention is enhanced by fabricating/manufacturing embodiments from a elastic or semielastic material that is flexible enough to yield under pressure, yet partially rigid, to maintain its original configuration in the absence of manual pressure, or equivalent pressure, that would otherwise deform the original configuration. The oral appliance 10 should resist a distorting influence and return to it's original size and shape when the influence is removed. Suitable materials could be selected from rubber-based materials, various silicones, including soft silicones, polyolefins (for example, high- and low-density polyethylene and high- and low-density polypropylene), synthetic resins commonly used to form castings and impressions for dental and other purposes, all of which must have an FDA-approved rating, and must be bisphenol A-free.

### Methods of Making an Oral Appliance of the Present Invention:

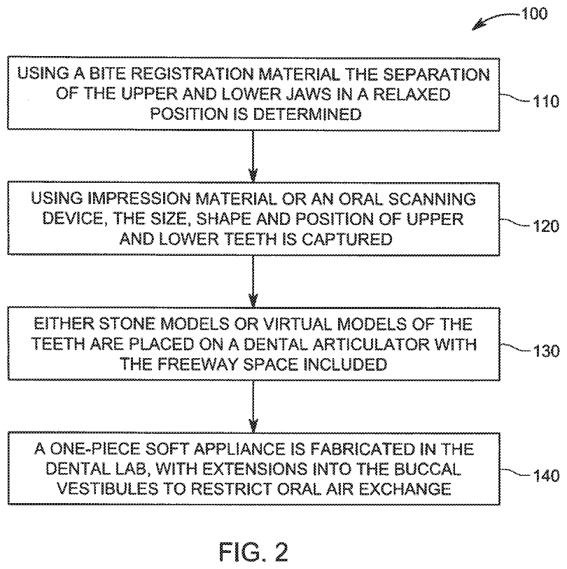

Referring next to FIG. 2, a method 100 for making an oral appliance 10 used for promoting nasal breathing while suppressing the oral breathing of a person 19 shall now be described in detail. The method 100 comprises a series of steps.

The first step 110 involves using a bite registration material that is configured for determining a separation value (also referred to as a “freeway space” within this patent specification) between a lower jaw 20A and an upper jaw 20B of the person 19. The separation value is determined when the lower jaw 20A and the upper jaw 20B are in the relaxed/retruded position, relative to each other. The separation value (sometimes referred to the freeway space) is usually between 5 and 15 mm.

A second step 120 involves using either an oral scanning device or an impression material fabricated, adapted, and configured to capture a size, a shape, and a position of each tooth in the lower and upper jaws 20A, 20B of the person 19, when in the relaxed position.

A third step 130 involves placing a model of the teeth of the person 19 (e.g., using either a stone model associated with the impression material or a virtual model for the oral scanning device) on a dental articulator that is adapted and configured to account for the separation value, as the articulator is adjustable and when a relaxed bite registration is captured in the mouth, it can be fitted to the plaster teeth on the articulator to duplicate that position of the jaws.

A fourth step 140 involves fabricating, preferably in a dental lab, the one-piece oral appliance of the present subject matter, in such a way as to define unitary extensions into the buccal vestibules of the person 19 to restrict oral air exchange when breathing orally.

### The Oral Cavity

The mouth consists of two regions: the vestibule and the oral cavity proper. The vestibule is the area between the teeth, the lips, and the cheeks. The oral cavity is bounded at the sides and in front by the so-called “alveolar process” (containing the teeth) and at the back by the isthmus of the fauces. Its roof is formed by the hard palate at the front, and the soft palate at the back. The uvula projects downwards from the middle of the soft palate at its back. The floor is formed by the mylohyoid muscles and is occupied mainly by the tongue. A mucous membrane—the oral mucosa, lines the sides and under surface of the tongue to the gums, and lines the inner aspect of the jaw (mandible). It receives secretions from the submandibular and sublingual salivary glands.

### Nerve Supply

The teeth and the periodontium (tissues that support the teeth) are innervated by the maxillary and mandibular nerves-divisions of the trigeminal nerve. Maxillary (upper) teeth and their associated periodontal ligament are innervated by the superior alveolar nerves, branches of the maxillary division, termed the posterior superior alveolar nerve, anterior superior alveolar nerve, and the variably present middle superior alveolar nerve. These nerves form the superior dental plexus above the maxillary teeth. The mandibular (lower) teeth and their associated periodontal ligament are innervated by the inferior alveolar nerve, a branch of the mandibular division. This nerve runs inside the mandible, in the inferior alveolar canal below the mandibular teeth, giving off branches to all the lower teeth (inferior dental plexus). The oral mucosa of the gingiva (gums) on the facial (labial) aspect of the maxillary incisors, canines and premolar teeth is innervated by the superior labial branches of the infraorbital nerve. The posterior superior alveolar nerve supplies the gingiva on the facial aspect of the maxillary molar teeth. The gingiva on the palatal aspect of the maxillary teeth is innervated by the greater palatine nerve apart from in the incisor region, where it is the nasopalatine nerve (long sphenopalatine nerve). The gingiva of the lingual aspect of the mandibular teeth is innervated by the sublingual nerve, a branch of the lingual nerve. The gingiva on the facial aspect of the mandibular incisors and canines is innervated by the mental nerve, the continuation of the inferior alveolar nerve emerging from the mental foramen. The gingiva of the buccal (cheek) aspect of the mandibular molar teeth is innervated by the buccal nerve (also called “long buccal nerve”).

### Dental Articulator

An articulator is a mechanical hinged device used in dentistry to which plaster casts of the maxillary (upper) and mandibular (lower) jaw are fixed, reproducing some or all the movements of the mandible in relation to the maxilla. The human maxilla is fixed and the scope of movement of the mandible (and therefore the dentition) is dictated by the position and movements of the bilateral temperomandibular joints, which sit in the glenoid fossae in the base of the skull. The temperomandibular joints are not a simple hinge but rotate and translate forward when the mouth is opened. The principal movements reproduced include: (1) at rest (centric jaw relation)—i.e., the retruded/relaxed position, (2) in protrusion (to bite), (3) from side to side (lateral excursion) to chew, (4) in retrusion, and (5) any possible combination of these. Counter-intuitively, it is the cast of the maxilla which moves relative to the cast of the mandible and the articulator. An articulator assists in the accurate fabrication of the biting surfaces of removable prosthodontic appliances (e.g., dentures), fixed prosthodontic restorations (implants, crowns, bridges, inlays, and onlays) and orthodontic appliances. When used with skill it ensures correct interdigitation of the teeth and an anatomically functional biting plane. This means less occlusal adjustments before and after fitting dental appliances and fewer chronic conflicts between teeth and jaw joints. Articulators are used mainly by dental technicians in fabrication of prostheses.

### Dental Lab

Dental labs manufacture or customize dental products to assist in the provision of oral health care by licensed dentists. These products include crowns, bridges, dentures, and other dental products. Dental lab technicians follow prescriptions from licensed dentists when fabricating such products, which include such prosthetic devices as denture teeth and implants, and therapeutic devices (e.g., orthodontic devices). Because the FDA regulates these dental products as medical devices, they are therefore subject to good manufacturing practice (“GMP”) and quality system (“QS”) standards of the FDA. Thus, in most cases, they are exempt from manufacturer registration oversight. Common restorations fabricated include crowns, bridges, dentures, and dental implants. Dental implants are one of the most advanced dental technologies in the field of dentistry.

Described in detail in this patent specification is an exemplary oral appliance designed for promoting nasal breathing and suppressing oral breathing of a person, and an exemplary method for making the oral appliance. While the present subject matter has been described in detail with reference to these embodiments, the present subject matter is not limited to these embodiments. On the contrary, many alternatives, changes, and/or modifications will become apparent to a person of ordinary skill in the prior art (“POSITA”) this patent specification and its associated figures are reviewed. Therefore, alternatives, changes, and/or modifications are to be considered as forming a part of the present subject matter—insofar as they fall within the spirit and scope of the appended claims.

A wearer (or “patient”) would only wear the appliance during sleep. After sleeping, the appliance would be cleaned and stored in a special container. The appliance of the present subject matter is preferably made by licensed dental professionals using dental laboratories licensed to fabricate the appliance. A one-way air valve could be incorporated into the design of the custom appliance to allow exhalation through both mouth and nose.

As used in this application, the term “about” or “approximately” refers to a range of values within plus or minus 10% of the specified number. And the term “substantially” refers to up to 80% or more of an entirety. Recitation of ranges of values herein are not intended to be limiting, referring instead individually to any and all values falling within the range, unless otherwise indicated, and each separate value within such a range is incorporated into the specification as if it were individually recited herein.

For purposes of this disclosure, the term “aligned” means parallel, substantially parallel, or forming an angle of less than 35.0 degrees. For purposes of this disclosure, the term “transverse” means perpendicular, substantially perpendicular, or forming an angle between 55.0 and 125.0 degrees. Also, for purposes of this disclosure, the term “length” means the longest dimension of an object. Also, for purposes of this disclosure, the term “width” means the dimension of an object from side to side. For the purposes of this disclosure, the term “above” generally means superjacent, substantially superjacent, or higher than another object although not directly overlying the object. Further, for purposes of this disclosure, the term “mechanical communication” generally refers to components being in direct physical contact with each other or being in indirect physical contact with each other where movement of one component affect the position of the other.

The use of any and all examples, or exemplary language (“e.g.,” “such as,” or the like) provided herein, is intended merely to better illuminate the embodiments and does not pose a limitation on the scope of the embodiments or the claims. No language in the specification should be construed as indicating any unclaimed element as essential to the practice of the disclosed embodiments.

In the following description, it is understood that terms such as “first,” “second,” “top,” “bottom,” “up,” “down,” and the like, are words of convenience and are not to be construed as limiting terms unless specifically stated to the contrary.

It should be understood, of course, that the foregoing relates to exemplary embodiments of the invention and that modifications may be made without departing from the spirit and scope of the invention as set forth in the following claims.

What is claimed is:

1. An oral appliance for promoting nasal breathing and for suppressing oral breathing of a wearer, the oral appliance comprising:
an upper flange providing a plurality of upper teeth impressions dimensioned and shaped to snugly receive the upper teeth of the wearer;
a lower flange unitary with the upper flange, wherein the lower flange providing a plurality of lower teeth impressions dimensioned and shaped to snugly receive the lower teeth of the wearer with their lower jaw in a retruded position; and
a closed wall extending between said flanges so that the closed wall prevents passage of an airflow between said flanges.

2. The oral appliance of claim 1, further comprising an upper spacer and a lower spacer, each spacer unitary with each upper flange and the lower flange, respectively, wherein each spacer anteriorly protrudes relative said flanges.

3. The oral appliance of claim 2, wherein the closed wall is posteriorly recessed relative to the spacers.

4. The oral appliance of claim 3, further comprising lateral spacer portions that interconnect the upper and lower spacers so as to define a closed loop.

5. The oral appliance of claim 4, wherein the spacers and lateral spacer portions are semicircular having a radius of between one and two millimeters.

6. The oral appliance of claim 1, further comprising an upper buccal vestibule barrier extending superiorly from the upper flange; and a lower buccal vestibule barrier extending inferiorly from the lower flange, wherein each buccal vestibule barrier is configured to occupy a buccal vestibule adjacent thereto so as to prevent passage of the airflow through the respective buccal vestibule.

7. The oral appliance of claim 1, further comprising:
an upper spacer and a lower spacer, each spacer unitary with each upper flange and the lower flange, respectively, wherein each spacer anteriorly protrudes relative said flanges, wherein the closed wall is posteriorly recessed relative to the spacers;
lateral spacer portions that interconnect the upper and lower spacers so as to define a closed loop, wherein the spacers and lateral spacer portions are semicircular having a radius of between one and two millimeters; and
an upper buccal vestibule barrier extending posteriorly from the upper flange; and a lower buccal vestibule barrier extending inferiorly from the lower flange, wherein each buccal vestibule barrier is configured to occupy a buccal vestibule adjacent thereto so as to prevent passage of the airflow through the respective buccal vestibule.

8. An oral appliance for promoting nasal breathing and for suppressing oral breathing of a wearer, the oral appliance comprising:
a unitary construction that occupies the oral cavity to prevent passage of an airflow from an external environment to the pharyngeal passageway of the wearer by way of the mouth of the wearer when the mandible is in a retruded position.

9. The oral appliance of claim 8, further comprising:
a plurality of impressions within the oral appliance for receiving upper and lower teeth of the wearer; and
upper and lower barriers that occupy the superior and inferior buccal vestibules, respectively, of the wearer.

10. The oral appliance of claim 9, further comprising anteriorly protruding upper and lower spacers for abutting an inner surface of the upper and lower lips of the wearer so that a distal edge of said lips sealably contact when the mandible is in the retruded position.

11. A method of making a unitary oral appliance of claim 7, the method comprising:
using a bite registration material configured for determining a separation value between a lower jaw and an upper jaw of the wearer, wherein the separation value is determined when the lower jaw and the upper jaw are in a relaxed position relative to each other;
using an impression material configured to capture a size, a shape, and a position of each tooth in the lower and upper jaws of the wearer;
placing a model of the teeth of the wearer on a dental articulator adapted and configured to account for the separation value; and
fabricating the unitary oral appliance having extensions into the buccal vestibules of the wearer to restrict oral air exchange when breathing orally.

12. The method of claim 11, wherein the model is a stone model.

13. The method of claim 12, wherein the fabricating step occurs in a dental lab.

14. A method of making a unitary oral appliance of claim 7, the method comprising:
using a bite registration material configured for determining a separation value between a lower jaw and an upper jaw of the wearer, wherein the separation value is determined when the lower jaw and the upper jaw are in a relaxed position relative to each other;
using an oral scanning device configured to capture a size, a shape, and a position of each tooth in the lower and upper jaws of the wearer;
placing a model of the teeth of the wearer on a dental articulator adapted and configured to account for the separation value; and
fabricating the unitary oral appliance having extensions into the buccal vestibules of the wearer to restrict oral air exchange when breathing orally.

15. The method of claim 14, wherein the model is a virtual model.

16. The method of claim 15, wherein the fabricating step occurs in a dental lab.
